(19) United States
(12) Patent Application Publication
(10) Pub. No.: US 2026/0287595 A1
(43) Pub. Date: Sep. 24, 2026

(54) MOLECULAR ANALYSIS OF EXTRACELLULAR VESICLES (EVs) FOR THE DETECTION OF IDIOPATHIC PULMONARY FIBROSIS

(71) Applicant: The General Hospital Corporation, Boston, MA (US)

(72) Inventors: Hyungsoon Im, Boston, MA (US); Miho Jeong, Boston, MA (US)

(21) Appl. No.: 19/469,796

(22) PCT Filed: Mar. 27, 2024
(86) PCT No.: PCT/US2024/021746

§ 371 Date: Sep. 26, 2025

Related U.S. Application Data
(60) Provisional application No. 63/492,254, filed on Mar. 27, 2023.

(51) Int. Cl. G01N 33/68; G01N 33/58

(52) U.S. Cl. CPC G01N 33/6884; G01N 33/582; G01N 2800/12

(57) ABSTRACT

Provided herein are method for detecting idiopathic pulmonary fibrosis in a subject. Additionally provided are methods of diagnosing and optionally treating a subject with idiopathic pulmonary fibrosis. The methods include enriching a sample for extracellular vesicles (EVs) and determining whether idiopathic pulmonary fibrosis marker(s) and cell-specific marker(s) co-localize within a sample, and further if the co-localization occurs at or beyond a particular reference level, the subject is diagnosed with idiopathic pulmonary fibrosis.

### CLAIM OF PRIORITY

This application claims the benefit of U.S. Provisional Application Ser. No. 63/492,254, filed on Mar. 27, 2023. The entire contents of the foregoing are incorporated herein by reference.

### FEDERALLY SPONSORED RESEARCH OR DEVELOPMENT

This invention was made with Government support under Grant No. GM138778 awarded by the National Institutes of Health. The Government has certain rights in the invention.

### TECHNICAL FIELD

Diagnostic methods for the detection of idiopathic pulmonary fibrosis.

### BACKGROUND

Extracellular vesicles (EVs) serve as surrogate markers of cellular activity, garnering attention as potential biomarkers due to their ubiquitous secretion by most cell types and presence of biomolecules reflective of their cellular origins. However, the efficacy of single EV analysis systems in distinguishing EVs based on their marker positivity and cellular origins. This requires multiplexed analysis at a single EV level and robust signal amplification systems to mitigate weak signals, which remains unclear. Despite the ability to detect single EVs, the application of such techniques has thus far mirrored that of bulk EV assays, and identifying diseases that may benefit from single EV analysis has proven challenging.

### SUMMARY

Provided herein are non-invasive methods of detecting idiopathic pulmonary fibrosis in a subject, comprising (a) isolating, detecting, or enriching extracellular vesicles (EVs) from a sample obtained from a subject; (b) labeling the EVs with one or more antibodies or antigen binding portions thereof that bind to one or more idiopathic pulmonary fibrosis marker(s) and one or more antibodies or antigen binding portions thereof that bind to one or more cell-specific marker(s), preferably wherein the markers are as shown in Table 1; (c) determining the counts of EVs positive for colocalized idiopathic pulmonary fibrosis marker(s) and cell-specific marker(s) or determining the intensity levels of idiopathic pulmonary fibrosis marker(s) and cell-specific marker(s) expressed in EVs.

In some embodiments, the methods include using a plasmon-enhanced EV detection method in step (b). In some embodiments, the antibodies or antigen binding portions thereof that bind to the EV idiopathic pulmonary fibrosis marker(s) comprise fluorescent dyes. In some embodiments, the one or more antibodies or antigen binding portions thereof that bind the one or more idiopathic pulmonary fibrosis marker(s) comprise one or more antibodies or antigen binding portions thereof that bind to matrix metallopeptidase 7 (MMP-7), cysteine-rich intestinal protein (CRIP-1), tubulin polymerization promoting protein family member 3 (TPPP3), calcium/calmodulin-dependent protein kinase 1 (CAMPK1D), gelsolin (GSN), collagen, type I, alpha 1 (Col1A1), pro-alpha1 chains of type III collagen (COL3A1), plasminogen activator inhibitor 1 (SERPINE1), triggering receptor expressed on myeloid cells 2 (TREM2), chemokine ligand 18 (CCL18), secreted phosphoprotein 1 (SPP-1), alpha-smooth muscle actin (α-SMA), and/or CD206. In some embodiments, the one or more antibodies or antigen binding portions thereof that bind the one or more cell-specific marker(s) comprise one or more antibodies or antigen binding portions thereof that bind to epithelial cellular adhesion molecule (EpCAM), E-cadherin, platelet-derived growth factor receptor alpha (PDGFRα), fibroblast activation protein (FAP), CD45, and/or CD68. In some embodiment, the determining the counts of EVs positive for of idiopathic pulmonary fibrosis marker(s) and cell-specific marker(s) comprises quantification of expression, concentration, intensity, or colocalization. In some embodiments, the quantification of expression, concentration, intensity, or colocalization are analyzed using multichannel fluorescence imaging in a single EV. In some embodiments, the sample comprises bronchoalveolar lavage fluid.

Provided herein are non-invasive methods of diagnosing idiopathic pulmonary fibrosis in a subject comprising: (a) isolating, detecting, or enriching extracellular vesicles (EVs) from a sample obtained from a subject, preferably wherein the EVs are labeled with one or more antibodies or antigen binding portions thereof that bind to one or more idiopathic pulmonary fibrosis marker(s) and one or more antibodies or antigen binding portions thereof that bind to one or more cell-specific marker(s), preferably wherein the markers are as shown in Table 1; (b) determining the counts of EVs positive for colocalized idiopathic pulmonary fibrosis marker(s) and cell-specific marker(s) or determining the intensity levels of idiopathic pulmonary fibrosis marker(s) and cell-specific marker(s) expressed in EVs; and (c) comparing the counts of EVs or the marker intensity levels determined in step (b) to a reference level, wherein the counts of EVs or the marker intensity levels determined in step (b) greater than the reference level indicate whether the subject has idiopathic pulmonary fibrosis.

In some embodiments, the methods include using a plasmon-enhanced EV detection method in step (b). In some embodiments, the antibodies or antigen binding portions thereof that bind to the EV idiopathic pulmonary fibrosis marker(s) comprise fluorescent dyes. In some embodiments, the one or more antibodies or antigen binding portions thereof that bind the one or more idiopathic pulmonary fibrosis marker(s) comprise one or more antibodies or antigen binding portions thereof that bind to matrix metallopeptidase 7 (MMP-7), cysteine-rich intestinal protein (CRIP-1), tubulin polymerization promoting protein family member 3 (TPPP3), calcium/calmodulin-dependent protein kinase 1 (CAMPK1D), gelsolin (GSN), collagen, type I, alpha 1 (Col1A1), pro-alpha1 chains of type III collagen (COL3A1), plasminogen activator inhibitor 1 (SERPINE1), chemokine ligand 18 (CCL18), triggering receptor expressed on myeloid cells 2 (TREM2), secreted phosphoprotein 1 (SPP-1), alpha-smooth muscle actin (α-SMA), and/or CD206, preferably MMP7, SPP1, and/or Col1A1. In some embodiments, the one or more antibodies or antigen binding portions thereof that bind the one or more cell-specific marker(s) comprise one or more antibodies or antigen binding portions thereof that bind to EpCAM, E-cadherin, PDGFRα, FAP, CD45, and/or CD68. In some embodiments, the determining the counts of EVs positive for idiopathic pulmonary fibrosis marker(s) and cell-specific marker(s) comprises quantification of expression, concentration, intensity, or colocalization. In some embodiments, the quantification of expression, concentration, intensity, or colocalization are analyzed using multichannel fluorescence imaging in a single EV. In some embodiments, the sample comprises bronchoalveolar lavage fluid.

Provided herein are methods for monitoring idiopathic pulmonary fibrosis in a subject over time, the method comprising: (a) isolating extracellular vesicles (EVs) in a first sample obtained from a subject at a first time point, wherein isolating EVs comprises: (i) applying the first sample to a functionalized substrate to capture extracellular vesicles (EVs); (ii) labeling the EVs with an antibody or antigen binding portions thereof that bind to a previously selected idiopathic pulmonary fibrosis marker(s); and (iii) labeling the EVs with an antibody or antigen binding portions thereof that bind to previously a selected cell-specific marker(s); preferably wherein the idiopathic pulmonary fibrosis markers and cell-specific markers are as shown in Table 1, (b) determining a count of EVs positive for the co-localization of cell-specific marker(s) and idiopathic pulmonary fibrosis marker(s) at a first time point; (c) isolating EVs in a second sample from the subject obtained at a second time point, wherein the isolating EVs comprises: (i) applying the second sample to a functionalized substrate to capture EVs; (ii) labeling the EVs with an antibody or antigen binding portions thereof that bind to the previously selected idiopathic pulmonary fibrosis marker(s) of step (a); and (iii) labeling the EVs with an antibody or antigen binding portions thereof that bind to previously a selected cell-specific marker(s) of step (a); (d) determining a count of EVs positive for the co-localization cell-specific marker(s) and idiopathic pulmonary fibrosis marker(s) at a second time point; thereby monitoring idiopathic pulmonary fibrosis in a subject over time.

In some embodiments, the methods include using a plasmon-enhanced EV detection method in step (b) and/or step (d). In some embodiments, the antibodies or antigen binding portions thereof that bind to the EV idiopathic pulmonary fibrosis marker(s) comprise fluorescent dyes. In some embodiments, the one or more antibodies or antigen binding portions thereof that bind the one or more idiopathic pulmonary fibrosis marker(s) comprise one or more antibodies or antigen binding portions thereof that bind to matrix metallopeptidase 7 (MMP-7), cysteine-rich intestinal protein (CRIP-1), tubulin polymerization promoting protein family member 3 (TPPP3), calcium/calmodulin-dependent protein kinase 1 (CAMPK1D), gelsolin (GSN), collagen, type I, alpha 1 (Col1A1), pro-alpha1 chains of type III collagen (COL3A1), plasminogen activator inhibitor 1 (SERPINE1), chemokine ligand 18 (CCL18), triggering receptor expressed on myeloid cells 2 (TREM2), secreted phosphoprotein 1 (SPP-1), alpha-smooth muscle actin (α-SMA), and/or CD206, preferably MMP7, SPP1, and/or Col1A1. In some embodiments, the one or more antibodies or antigen binding portions thereof that bind the one or more cell-specific marker(s) comprise one or more antibodies or antigen binding portions thereof that bind to EpCAM, E-cadherin, PDGFRα, FAP, CD45, and/or CD68. In some embodiments, the determining the counts of EVs positive for idiopathic pulmonary fibrosis marker(s) and cell-specific marker(s) comprises quantification of expression, concentration, intensity, or colocalization. In some embodiments, the quantification of expression, concentration, intensity, or colocalization are analyzed using multichannel fluorescence imaging in a single EV. In some embodiments, the sample comprises bronchoalveolar lavage fluid. In some embodiments, the presence of a level of EVs with co-localization of idiopathic pulmonary fibrosis marker(s) and cell-specific marker(s) above a reference level indicates that the subject has idiopathic pulmonary fibrosis. In some embodiments, the methods include identifying a subject has having a level of EVs with co-localization of idiopathic pulmonary fibrosis marker(s) and cell-specific marker(s) above a reference level and administering a treatment for idiopathic pulmonary fibrosis to the subject. In some embodiments, the methods include administration of supplemental oxygen therapy, an antifibrotic therapy (optionally pirfenidone) and a tyrosine kinase inhibitor (optionally nintedanib). In some embodiments, the methods include administering at least one dose of a treatment or candidate treatment for idiopathic pulmonary fibroses to the subject between steps (b) and (c). In some embodiments, the methods include administration of supplemental oxygen therapy, an antifibrotic therapy (optionally pirfenidone) and a tyrosine kinase inhibitor (optionally nintedanib).

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-K. Plasmon-enhanced multiplexed single EV imaging system distinguishes extracellular vesicles based on cellular origins. FIG. 1A is a multi-channel fluorescence microscopic image of macrophages' EVs derived from differentiated THP-1 by phorbol 12-myristate 13-acetate, captured on a nanoplasmonic sensing chip and labeled by TFP-AF488 and cell-specific markers PDGFRα (AF555) and CD45 (AF647) (top). The line profiles of fluorescence intensity through the white dashed line show co-localization of EV signals (AF488) and immune-cell specific CD45 signals (top), as shown in the following curve graph (bottom). FIGS. 1B and 1C show single EV dot plots of EVs captured from MRC5 lung fibroblast and THP-1 monocyte cell lines labeled PDGFRα (AF555) and CD45 (AF647) using glass and nanoplasmonic chips (FIG. 1C). CD45 (AF647) is the marker for macrophage cell line THP-1, and PDGFRα (AF555) is the marker for lung fibroblast cell line MRC5. FIGS. 1D and 1H show the percentage of CD45 positive EVs (FIG. 1D) and the percentage of PDGFRα positive EVs (FIG. 1H) in the total EVs captured from THP-1 or MRC5 cells using glass or nanoplasmonic sensor. The detection threshold was changed to monitor the detection variation of CD45 and PDGFRα in each type of EVs. FIG. 1E shows the ratio of CD45+ EVs, calculated by dividing the percentage of CD45+ EVs derived from THP-1 cells (as shown in FIG. 1D) by the percentage of CD45+ EVs derived from MRC5 cells (as shown in FIG. 1H). The EVs were captured on either glass or nanoplasmonic sensor. FIG. 1I shows the ratio of PDGFRα+ EV, calculated by dividing the percentage of CD45+ EVs derived from MRC5 cells (as shown in FIG. 1H) by the percentage of CD45+ EVs derived from THP-1 cells (as shown in FIG. 1D). The EVs were captured on either glass or nanoplasmonic sensor. FIGS. 1F and 1J show the validation of the detection thresholds for each cell specific marker using IgG isotype antibodies, AF647 (Ms IgG) (FIG. 1F) and AF555 (Rb IgG) (FIG. 1J) on AF488 labeled EVs. The EVs were captured using either glass or nanoplasmonic sensor. FIG. 1G shows the percentage of CD45+ EVs and CD45+ EV/CD45-EV in the total EVs captured by glass or nanoplasmonic sensor. FIG. 1K shows the percentage of PDGFRα+ EVs and PDGFRα+ EV/PDGFRα-EV in the total EVs captured by glass or nanoplasmonic sensor.

FIGS. 2A-2G. Specificity of cell-specific markers in EVs. FIG. 2A is a schematic depicting the lung epithelial niche, where epithelial cells (EpCAM+), fibroblasts (PDGFRα+), and macrophages (CD45+/CD68+) are located. These cell types communicate with each other and play key roles in the development of idiopathic pulmonary fibrosis (IPF). FIGS. 2B and 2C are 3D graphs showing the intensity of markers, EpCAM and CD45, on EVs captured by glass (FIG. 2B) or nanoplasmonic sensor (FIG. 2C). EVs were derived from BEAS-2B, MRC5, and THP-1 cell lines. The BEAS-2B cell line represents bronchial epithelial cells, the MRC5 cell line represents lung fibroblasts, and the THP-1 cell line represents macrophages. Each type of EVs were incubated with the antibody cocktails including anti-EpCAM (Cy7), anti-PDGFRα (AF555), and anti-CD45 (AF647) on glass or nanoplasmonic sensor. FIGS. 2D and 2E show the specificity of the antibodies in recognizing specific markers present in the EVs that were captured from BEAS-2B, MRC5, and THP-1 cell lines using either nanoplasmonic sensor (FIG. 2D) or beads-based flow cytometry (FIG. 2E). Each type of EV carries its distinctive markers: EpCAM for BEAS-2B EVs, PDGFRα for MRC5 EVs, and CD45 for THP-1 EVs. FIG. 2F shows the correlation of each cell marker measured in the nanoplasmonic sensor group and the bead-based flow cytometry group. The cell markers include EpCAM, CD45, and PDGFRα. The nanoplasmonic sensor group and bead-based flow cytometry group represent EVs captured using nanoplasmonic sensor and bead-based flow cytometry respectively. FIG. 2G is a graph depicting mixed EVs of two types derived from two different parental cells at various ratios, with an evaluation of cell-specific markers, including EpCAM, CD45, and PDGFRα. FIGS. 2H-2J show the counts of EpCAM+EV (FIG. 2H), PDGFRα+ EV (FIG. 2I), and CD45+ (FIG. 2J) in an EV mixture formed by combining two types of EVs derived from two different parental cells at various ratios as described in FIG. 2G.

FIGS. 3A-3C. Analysis of double-positive macrophage EVs. FIG. 3A shows dual marker analysis among an EV population. The EVs were double labeled with antibodies for macrophage cell-specific marker CD68+ (AF647) and PF marker fibronectin (AF555). The EV location information was firstly collected by EV channel (Cy7) prior to dual marker analysis. M2 EVs are EVs derived from M2-like macrophages. MO (FIG. 3B) and M2 EVs (FIG. 3C) show the dot plots plotting with the intensity of AF555 (FN+) and intensity of AF647 (CD68+). The first quadrant represents double-positive EVs, the second quadrant represents IPF marker FN+ EVs, and the fourth quadrant represents cell-specific marker CD68+ EVs.

FIGS. 4A-4E. Analysis of double-positive epithelial, macrophage, and fibroblast EVs. FIG. 4A is a schematic showing the selected IPF markers for each cell type including epithelial cells, fibroblasts, and macrophages. MMP-7, Col1A1, and SPP-1 were the most intersected markers for epithelial cells, fibroblasts, and macrophages, respectively. FIG. 4B is a diagram showing marker-overexpression by in vitro cell culture. BEAS-2B cells were exposed to TGF-β for 48 hours to induce epithelial-mesenchymal transition (EMT). MRC5 cells were exposed to TGF-β for 48 hours to induce fibroblast-myofibroblast transition (FMT). THP-1 cells were stimulated with IL-4 and IL-13 for 48 hours to generate M2-like macrophages. FIGS. 4C-4D show the intensity of the fibroblast IPF marker Col1A1 (AF647) in MRC5 cells not treated with TGF-β (FIG. 4C) and FMT after TGF-β stimulation (FIG. 4D). FIG. 4E shows the percentage of EVs that were single positive for fibroblast IPF marker Col1A1 and the percentage of EVs that were double positive for Col1A1 and fibroblast marker PDGFRα in FMT compared to control cells (non-treated cells). In addition, FIG. 4E shows the intensity of double-positive EVs (Col1A1+PDGFRα+) derived from FMT compared to those derived from control cells. FIG. 4F shows the differential expression of IPF markers for each cell type, including FN and MMP7 for epithelial cells (BEAS-2B), FN and Col1A1 for fibroblasts (MRC5), and FN and SPP-1 for macrophages (THP-1), before and after the induction by TGF-β or IL-4/IL-13 as described in FIG. 4B. The percentage of EVs that were single positive for each of above IPF markers was measured in corresponding cells before and after the induction. The percentage of EVs that were double positive for one of the IPF markers and a cell specific marker was measured in corresponding cells before and after the induction. The intensity of double-positive EVs (IPF marker positive and cell specific marker positive) was measured in corresponding cells before and after the induction. The value of the treated groups were divided by the value of the non-treated groups such that the size of the circle represents a fold induction of treated over non-treated conditions.

FIG. 5 is a diagram showing the scheme of 3-channel single EV analysis, where EVs are labeled by Cy7, cell-specific markers are labeled by AF555, and IPF markers are labeled by AF647. EVs captured by nanoplasmonic sensor were targeted with the primary antibodies for IPF makers (e.g., FN) and the primary antibodies for cell-specific markers (e.g., EpCAM). Subsequently, AF647 or AF555 conjugated secondary antibodies were applied to visualize the marker specific EVs.

FIGS. 6A-6D show the process of isolating and labeling BALF EVs acquired from IPF patients. BALF samples were collected from five selected IPF patients (FIG. 6A). The BALF EVs were captured using size exclusion chromatography (FIG. 6B). The captured BALF EVs were fluorescently labeled for EV quantification (FIG. 6C). EV and cell-specific markers were measured and represented as a heatmap after quantification (FIG. 6D).

### DETAILED DESCRIPTION

Described herein are methods capable of detecting cell-specific EVs with high specificity and evaluating idiopathic pulmonary fibrosis (IPF) markers from each cell-specific EV using a multiplexed system. IPF involves the interplay of epithelial cells, macrophages, and lung fibroblasts, each expressing distinct markers, and the EVs of the methods are derived from these cell types. The methods herein not only detect cell-specific markers but also IPF-associated markers, providing insight into the status of cells involved in fibrogenesis. Bronchoalveolar lavage fluid (BALF) from IPF patients can be assessed, facilitating disease diagnosis and revealing correlation with marker expression in tissue samples.

### Idiopathic Pulmonary Fibrosis

Idiopathic pulmonary fibrosis (IPF) is a lung disease wherein scar tissue grows inside the lungs of a subject, making it difficult to breathe. The condition gets worse over time. While the precise cause of IPF is often unexplained, risk factors include things that irritate or attack the lungs, like cigarette smoking, inhalation of wood dust or asbestos, or infections such as the flu, Epstein-Barr virus, hepatitis, and herpes. Current tests for IPF include chest scans such as x-rays or high-resolution computed tomography, breathing into a spirometer, pulse oximetry, blood tests to check for carbon dioxide levels. These tests are often not definitive.

Idiopathic pulmonary fibrosis (IPF) is an irreversible, fatal disease leading to organ failure. Even though accurate and early diagnosis is critical for patient treatment, there is no method for evaluating IPF progression precisely. Extracellular vesicles (EVs) in bronchoalveolar lavage fluid (BALF) play important roles as cellular surrogates for molecular IPF diagnosis. However, the current bulk EV analysis prevents the accurate evaluation of EV biomarkers derived from various cell types.

### Method of Detecting Idiopathic Pulmonary Fibrosis

The present methods can include isolating particular EV populations (e.g., epithelial, macrophage, or fibroblast EVs) in a subject suspected of having idiopathic pulmonary fibrosis (IPF) and measuring the EV expression of IPF biomarkers. Expression of IPF biomarkers can be measured over time to monitor the course and/or progression of IPF in a subject.

A subject can be an individual (e.g., a mammal such as a human) having or suspected of having IPF. In some embodiments, the subject can be receiving a type of IPF treatment (e.g., medicine such as nintedanib and/or pirfenidone, or oxygen therapy). A sample can be obtained from the subject. A sample can mean any sample, including, but not limited to cells, lysed cells, cellular extracts, nuclear extracts, extracellular fluid, media in which cells (e.g., bronchoalveolar lavage fluid from the subject) are cultured. In some embodiments, a sample is obtained from a subject at multiple time points.

The sample from the subject can be enriched for EVs, e.g., based on the presence of EV cell-specific markers. In some embodiments, the methods can include using antibodies or antigen binding portions thereof that bind to selected EV cell-specific markers in order to identify or enrich EVs for further analysis. For example, the antibodies can be capture antibodies that are attached to a substrate (e.g., a plate, well, or beads). A sample from a subject, e.g., a sample comprising a population of EVs (optionally EVs obtained from a biofluid such as bronchoalveolar lavage fluid from the subject) can then be applied to the substrate, wherein the antibodies or antigen binding portions thereof that bind to the selected EV cell-specific markers capture and enrich the EV population for EVs having the specified EV IPF markers. IPF markers can then be evaluated in the EVs, e.g., optionally using antibodies that bind the IPF markers. A sample from the subject, e.g., a sample comprising a population of EVs (optionally EVs obtained from a biofluid such as bronchoalveolar lavage fluid from the subject) can be applied to a nanoplasmonic EV sensing platform (e.g., nPLEX platform), which amplifies EVs' immunofluorescence signals for multi-color fluorescence imaging.

In some embodiments, the antibodies or antigen binding portions thereof that bind to the selected EV IPF markers (e.g., MMP-7, CRIP-1, TPPP3, CAMPK1D, GSN, Col1A1, COL3A1, SERPINE1, TREM2, CCL18, SPP-1, α-SMA, and/or CD206, preferably MMP-7, SPP-1, and/or Col1A1) can be applied to a sample, wherein the sample has been previously enriched for cell-specific EVs (e.g., EpCAM, E-cadherin, CD68, CD45, PDGFRα, and/or FAP). In some embodiments, the antibodies or antigen binding portions thereof that bind to the selected EV IPF markers (e.g., MMP-7, CRIP-1, TPPP3, CAMPK1D, GSN, Col1A1, COL3A1, SERPINE1, TREM2, CCL18, SPP-1, α-SMA, and/or CD206, preferably MMP-7, SPP-1, and/or Col1A1) can be applied as free antibodies to the EV sample, wherein the antibodies or antigen binding portions thereof that bind to the selected EV IPF markers (e.g., MMP-7, CRIP-1, TPPP3, CAMPK1D, GSN, Col1A1, COL3A1, SERPINE1, TREM2, CCL18, SPP-1, α-SMA, and/or CD206, preferably MMP-7, SPP-1, and/or Col1A1) can be labeled (e.g., fluorescently labeled) or wherein the antibodies or antigen binding portions thereof that bind to the selected EV IPF markers (e.g., MMP-7, CRIP-1, TPPP3, CAMPK1D, GSN, Col1A1, COL3A1, SERPINE1, TREM2, CCL18, SPP-1, α-SMA, and/or CD206, preferably MMP-7, SPP-1, and/or Col1A1) can be detected with a secondary antibody. In some embodiments, the antibodies or antigen binding portions thereof that bind to the selected EV IPF markers (e.g., MMP-7, CRIP-1, TPPP3, CAMPK1D, GSN, Col1A1, COL3A1, SERPINE1, TREM2, CCL18, SPP-1, α-SMA, and/or CD206, preferably MMP-7, SPP-1, and/or Col1A1) can be applied before, after, concurrently with the antibodies or antigen binding portions thereof that bind to cell-specific markers (e.g., EpCAM, E-cadherin, CD68, CD45, PDGFRα, and/or FAP).

In some embodiments, EVs from a sample can be enriched using a plasmon-enhanced EV capture method such as a nanoplasmonic exosome (e.g., nPLEX) sensor. See WO2015084800, which is incorporated by reference herein. In some embodiments, the plasmon-enhanced EV capture method includes EV capture using any substrate, e.g., plain substrate, nanostructures, beads, or other materials. Antibodies to EV IPF markers (e.g., MMP-7, CRIP-1, TPPP3, CAMPK1D, GSN, Col1A1, COL3A1, SERPINE1, TREM2, CCL18, SPP-1, α-SMA, and/or CD206, preferably MMP-7, SPP-1, and/or Col1A1) can be applied to the EV-cell-specific enriched sample, wherein the antibodies to EV IPF marker(s) can be labeled (e.g., fluorescently labeled) or wherein secondary antibodies can be used to detect the antibodies to EV IPF marker(s). Antibodies to cell-specific marker(s) (e.g., EpCAM, E-cadherin, CD68, CD45, PDGFRα, and/or FAP) can be applied to an EV-enriched sample, wherein the antibodies to the cell-specific marker(s) (e.g., EpCAM, E-cadherin, CD68, CD45, PDGFRα, and/or FAP) can be labeled (e.g., fluorescently labeled) or wherein secondary antibodies can be used to detect the antibodies to cell-specific marker(s). In some embodiments, the antibodies to EV IPF marker(s) (e.g., MMP-7, CRIP-1, TPPP3, CAMPK1D, GSN, Col1A1, COL3A1, SERPINE1, TREM2, CCL18, SPP-1, α-SMA, and/or CD206, preferably MMP-7, SPP-1, and/or Col1A1) and the antibodies to the cell-specific marker(s) (e.g., EpCAM, E-cadherin, CD68, CD45, PDGFRα, and/or FAP) can be applied to the sample and/or the EV-enriched sample at the same time. In some embodiments, the antibodies to the EV IPF marker(s) (e.g., MMP-7, CRIP-1, TPPP3, CAMPK1D, GSN, Col1A1, COL3A1, SERPINE1, TREM2, CCL18, SPP-1, α-SMA, and/or CD206, preferably MMP-7, SPP-1, and/or Col1A1) are applied to the sample and/or EV-enriched sample prior to the antibodies to the cell-specific marker(s) (e.g., EpCAM, E-cadherin, CD68, CD45, PDGFRα, and/or FAP) are applied to the sample and/or EV-enriched sample. In some embodiments, the antibodies to the EV IPF marker(s) (e.g., MMP-7, CRIP-1, TPPP3, CAMPK1D, GSN, Col1A1, COL3A1, SERPINE1, TREM2, CCL18, SPP-1, α-SMA, and/or CD206, preferably MMP-7, SPP-1, and/or Col1A1) are applied to the sample and/or EV-enriched sample after to the antibodies to the cell-specific marker(s) (e.g., EpCAM, E-cadherin, CD68, CD45, PDGFRα, and/or FAP) are applied to the sample and/or EV-enriched sample. In some embodiments, the co-localized IPF marker(s) and cell-specific marker(s) are assessed and detected. In some embodiments, the co-localized IPF marker(s) and cell-specific marker(s) are compared to a reference level.

The methods can include detecting the colocalization of the IPF markers in specific cell types, e.g., as shown in Table 1. In some embodiments of any of the methods described herein, at least two, three, four, five, six, seven, eight, nine, ten, 11, 12, or all 13 of the IPF markers are detected, e.g., at least one, two, three, four, or five IPF markers from each cell type as shown in Table 1, and at least one or two cell-specific markers from each cell type as shown in Table 1.

TABLE 1
IPF and Cell-Specific Markers

| | Epithelial cells | Macrophages | Fibroblasts |
|---|---|---|---|
| Cell-specific | EpCAM, | CD68, CD45 | PDGFRα, FAP |
| markers | E-cadherin | | |
| IPF markers | MMP7, CRIP-1, | SPP-1, TREM2, | COL1A1, COL3A1, |
| | CAMPK1D, | CCL18, CD206 | SERPINE1, α-SMA |
| | TPPP3, GSN | | |

### Method of Diagnosing Idiopathic Pulmonary Fibrosis

Included herein are methods for diagnosing idiopathic pulmonary fibrosis. The methods rely on detection of a co-localized biological marker or a plurality of biological makers of IPF (e.g., MMP-7, CRIP-1, TPPP3, CAMPK1D, GSN, Col1A1, COL3A1, SERPINE1, TREM2, CCL18, SPP-1, α-SMA, and/or CD206, preferably MMP-7, SPP-1, and/or Col1A1) with a biological marker or plurality of biological cell-specific marker(s) (e.g., EpCAM, E-cadherin, CD68, CD45, PDGFRα, and/or FAP), e.g., as shown in Table 1. The methods include obtaining a sample from a subject, and evaluating the presence and/or level of co-localized IPF marker(s) and cell-specific marker(s) in the sample, and comparing the presence and/or level with one or more references, e.g., a control reference that represents a normal level of co-localized IPF marker(s) and cell-specific marker(s), e.g., a level in an unaffected subject, and/or a disease reference that represents a level of the proteins associated with co-localized IPF marker(s) and cell-specific marker(s), e.g., a level in a subject not having IPF.

As used herein the term “sample”, when referring to the material to be tested for the presence of a biological marker (e.g., MMP-7, CRIP-1, TPPP3, CAMPK1D, GSN, Col1A1, COL3A1, SERPINE1, TREM2, CCL18, SPP-1, α-SMA, CD206, EpCAM, E-cadherin, CD68, CD45, PDGFRα, and/or FAP, preferably MMP-7, SPP-1, Col1A1, EpCAM, E-cadherin, CD68, CD45, PDGFRα, and/or FAP) using the method of the invention, includes inter alia tissue, whole blood, plasma, serum, urine, sweat, saliva, breath, exosome or exosome-like microvesicles (U.S. Pat. No. 8,901,284), lymph, feces, cerebrospinal fluid, ascites, bronchoalveolar lavage fluid, pleural effusion, seminal fluid, sputum, nipple aspirate, post-operative seroma or wound drainage fluid. The type of sample used may vary depending upon the identity of the biological marker to be tested and the clinical situation in which the method is used. Various methods are well-known within the art for the identification and/or isolation and/or purification of a biological marker from a sample. An “isolated” or “purified” biological marker is substantially free of cellular material or other contaminants from the cell or tissue source from which the biological marker is derived, i.e., partially or completely altered or removed from the natural state through human intervention. For example, nucleic acids contained in the sample are first isolated according to standard methods, for example using lytic enzymes, chemical solutions, or isolated by nucleic acid-binding resins following the manufacturer's instructions.

The presence and/or level of a protein can be evaluated using methods known in the art, e.g., using standard electrophoretic and quantitative immunoassay methods for proteins, including but not limited to, Western blot; enzyme linked immunosorbent assay (ELISA); biotin/avidin type assays; protein array detection; radio-immunoassay; immunohistochemistry (IHC); immune-precipitation assay; FACS (fluorescent activated cell sorting); mass spectrometry (Kim (2010) Am J Clin Pathol 134:157-162; Yasun (2012) Anal Chem 84 (14): 6008-6015; Brody (2010) Expert Rev Mol Diagn 10 (8): 1013-1022; Philips (2014) PLOS One 9 (3): e90226; Pfaffe (2011) Clin Chem 57 (5): 675-687). The methods typically include revealing labels such as fluorescent, chemiluminescent, radioactive, and enzymatic or dye molecules that provide a signal either directly or indirectly. As used herein, the term “label” refers to the coupling (i.e., physically linkage) of a detectable substance, such as a radioactive agent or fluorophore (e.g., phycoerythrin (PE) or indocyanine (Cy5), to an antibody or probe, as well as indirect labeling of the probe or antibody (e.g., horseradish peroxidase, HRP) by reactivity with a detectable substance.

In some embodiments, an IHC method may be used. IHC provides a method of detecting a biological marker in situ. The presence and exact cellular location of the biological marker can be detected. Typically, a sample is fixed with formalin or paraformaldehyde, embedded in paraffin, and cut into sections for staining and subsequent inspection by confocal microscopy. Current methods of IHC use either direct or indirect labelling. The sample may also be inspected by fluorescent microscopy when immunofluorescence (IF) is performed, as a variation to IHC.

In some embodiments, the methods herein provide for diagnosing a subject with idiopathic pulmonary fibrosis. Diagnosing a subject with IPF can include the methods of detecting IPF, and further include comparing the number, amount, or intensity of co-localized IPF marker(s) and cell-specific marker(s) to a reference level. If the co-localized IPF marker(s) and cell-specific marker(s) meet or exceed a reference level, the subject may be further given one or more treatments used for IPF. A level as used herein can include a ratio of EVs that comprise co-localized markers (e.g., as shown in Table 1) to EVs that do not comprise co-localized markers, e.g., a percentage of EVs with a given cell-specific marker that also include an IPF marker, out of the EVs with the a given cell-specific marker that do not also include an IPF marker.

In some embodiments, enriching EVs (e.g., epithelial, fibroblast, and/or macrophage EVs) and probing the EVs for levels of IPF marker(s) can be carried out at multiple time points (e.g., over time or longitudinally). For example, enriching EVs and probing the EVs for levels of IPF marker(s) and cell-specific marker(s) can be carried out at one, two, three, four, five, or more time points. In some embodiments, the level (as determined by antibody detection) of co-localized IPF marker(s) and cell-specific marker(s) at a first time point can be used to determine the relative level of co-localized IPF marker(s) and cell-specific marker(s) at a second time point by comparing the amount of co-localized IPF marker(s) and cell-specific marker(s) signal at the second time point to the co-localized IPF marker(s) and cell-specific marker(s) signal of the first time point and noting an increase or decrease of co-localized IPF marker(s) and cell-specific marker(s) signal. Similarly, the amount of co-localized IPF marker(s) and cell-specific marker(s) signal at a third (or fourth, or fifth, etc.) time point can be compared to the amount of co-localized IPF marker(s) and cell-specific marker(s) signal at the first time point, or the co-localized IPF marker(s) and cell-specific marker(s) signal at the third (or fourth, or fifth, etc.) time point can be compared to the amount of co-localized IPF marker(s) and cell-specific marker(s) signal at any previous time point to analyze whether there are any trends in co-localized IPF marker(s) and cell-specific marker(s) signal over time.

In some embodiments, a relative increase in levels of co-localized IPF marker(s) and cell-specific marker(s) EVs over a previous level of co-localized IPF marker(s) and cell-specific marker(s) EVs indicates the IPF in the subject is progressing in the disease (e.g., the IPF symptoms are getting worse). In some embodiments, a relative decrease or no significant change in levels of co-localized IPF marker(s) and cell-specific marker(s) EVs over the previous level of co-localized IPF marker(s) and cell-specific marker(s) EVs indicates IPF in the subject is not progressing.

The relative levels of co-localized IPF marker(s) and cell-specific marker(s) EVs thus can be used to determine whether the subject receives additional IPF treatments to manage the subject's IPF.

In some embodiments, a relative increase in co-localized IPF marker(s) and cell-specific marker(s) EVs over a previous level of co-localized IPF marker(s) and cell-specific marker(s) EVs indicates whether the subject should continue to receive IPF treatment. In some embodiments, a relative increase in co-localized IPF marker(s) and cell-specific marker(s) EVs over the previous level of co-localized IPF marker(s) and cell-specific marker(s) EVs indicates the subject should receive additional IPF treatments. In some embodiments, the subject only receives further IPF treatments if there is a no change or an increase in co-localized IPF marker(s) and cell-specific marker(s) EVs over the previous (or any previous) level of co-localized IPF marker(s) and cell-specific marker(s) EVs.

In some embodiments, the presence and/or level of co-localized IPF marker(s) and cell-specific marker(s) EVs is comparable or greater to the presence and/or level of the IPF reference level, and the subject has one or more symptoms associated with IPF, then the subject has IPF. In some embodiments, the subject has no overt signs or symptoms of IPF, but the presence and/or level of one or more of the co-localized IPF marker(s) and cell-specific marker(s) EVs evaluated is comparable to the presence and/or level of the co-localized IPF marker(s) and cell-specific marker(s) EVs in the IPF reference, then the subject has an increased risk of developing IPF. In some embodiments, once it has been determined that a person has IPF, or has an increased risk of developing IPF, then a treatment, e.g., as known in the art or as described herein, can be administered.

Suitable reference values can be determined using methods known in the art, e.g., using standard clinical trial methodology and statistical analysis. The reference values can have any relevant form. In some cases, the reference comprises a predetermined value for a meaningful level of co-localized IPF marker(s) and cell-specific marker(s) EVs, e.g., a control reference level that represents a normal level of co-localized IPF marker(s) and cell-specific marker(s) EVs, e.g., a level in an unaffected subject or a subject who is not at risk of developing IPF.

The predetermined level can be a single cut-off (threshold) value, such as a median or mean, or a level that defines the boundaries of an upper or lower quartile, tertile, or other segment of a clinical trial population that is determined to be statistically different from the other segments. It can be a range of cut-off (or threshold) values, such as a confidence interval. It can be established based upon comparative groups, such as where association with risk of developing disease or presence of disease in one defined group is a fold higher, or lower, (e.g., approximately 2-fold, 4-fold, 8-fold, 16-fold or more) than the risk or presence of disease in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk.

In some embodiments, the predetermined level is a level or occurrence in the same subject, e.g., at a different time point, e.g., an earlier time point.

Subjects associated with predetermined values are typically referred to as reference subjects. For example, in some embodiments, a control reference subject does not have a disorder described herein (e.g., IPF).

A disease reference subject is one who has (or has an increased risk of developing) IPF. An increased risk is defined as a risk above the risk of subjects in the general population.

The predetermined value can depend upon the particular population of subjects (e.g., human subjects) selected. For example, an apparently healthy population will have a different ‘normal’ range of levels of co-localized IPF marker(s) and cell-specific marker(s) EVs than will a population of subjects which have, are likely to have, or are at greater risk to have, a disorder described herein. Accordingly, the predetermined values selected may take into account the category (e.g., sex, age, health, risk, presence of other diseases) in which a subject (e.g., human subject) falls. Appropriate ranges and categories can be selected with no more than routine experimentation by those of ordinary skill in the art.

In characterizing likelihood, or risk, numerous predetermined values can be established.

Once a subject has been identified as having IPF using a method described herein, the methods can further include selecting and optionally administering a treatment for IPF to the subject. Treatments for IPF can include supplemental oxygen therapy, antifibrotic therapies (e.g., pirfenidone) and tyrosine kinase inhibitors (e.g., nintedanib), and combinations thereof.

### Extracellular Vesicles (EVs)

Extracellular vesicles (EVs) are lipid-based microparticles, nanoparticle, or protein-rich aggregates present in a sample (e.g., a biological fluid) obtained from a subject. Extracellular vesicles are also referred to in the art and herein as exosomes, microvesicles, or nanovesicles. Extracellular vesicles can include membrane vesicles secreted from cell surfaces (ectosomes), internal stores (exosomes), cancer cells (oncosomes), or released as a result of apoptosis and cell death. In addition to lipid membranes, depending on their cell or tissue of origin, EVs can include additional components such as lipoproteins, proteins, nucleic acids, phospholipids, amphipathic lipids, gangliosides and other particles contained within the lipid membrane or encapsulated by the EVs.

All cells likely release, secrete, or shed EVs, making them useful clinical diagnostic and therapeutic targets for a range of diseases. Non-limiting examples of normal or cancer cell types that can release EVs include liver cells (e.g., hepatocytes), lung cells, spleen cells, pancreas cells, colon cells, skin cells, bladder cells, eye cells, brain cells, esophagus cells, cells of the head, cells of the neck, cells of the ovary, cells of the testes, prostate cells, placenta cells, epithelial cells, endothelial cells, fibroblast cells, adipocyte cells, macrophages, kidney cells, heart cells, muscle cells, blood cells (e.g., white blood cells, platelets), and combinations of the foregoing. Because EVs are involved in cell-cell communication, their characterization casts light upon their role in normal physiology and pathology. EVs in biological fluids including saliva, urine, plasma, bronchoalveolar lavage fluid, and serum can be interrogated according to the methods described herein.

In some embodiments, an extracellular vesicle is between about 20 nm to about 200 nm in diameter. Individual EVs have ~ 1/10,000 the surface area and ~ 1/1,000,000 the volume of a whole cell and are therefore difficult to detect using single cell analysis tools, including conventional flow cytometry. As a result, most proteomic and genomic analysis is performed in bulk on thousands or millions of EVs. However, EVs in biofluids come from many different cell types, and from different locations from within the cell (exosomes secreted from intracellular multi-vesicular bodies, ectosomes/microvesicles shed from the plasma membrane surface, membrane fragments released as a result of cell apoptosis, necrosis, etc.). Thus, in a bulk analysis, the signature for a particular EVs may be lost in the background of vesicles from other sources, and methods of enriching EVs help capture a more robust EV picture.

EVs represent new opportunities as circulating cancer biomarkers. These cell-derived membrane-bound vesicles contain protein and nucleic acid cargo, providing a representative snapshot of the content of the secreting cells. Therefore, EVs can function as a novel biomarker for liquid biopsy in personalized medicine. However, EVs are relatively new targets for analytical assays in clinics and possess unique physical and biological traits. They fall in size range much smaller than cells but larger than proteins and exist in a highly heterogeneous biological background. These properties impose technical difficulties, which often lead to variable findings. Furthermore, identifying cell-specific (e.g., epithelial, fibroblast, macrophage origins) EVs and interrogating for idiopathic pulmonary fibrosis marks within the subpopulation require multiplexed analysis, ideally in a single EV resolution.

Molecular characterization of single EVs is technically challenging. Most EVs are small vesicles (<200 nm) with limited numbers of epitopes and surface areas for labeling (i.e., weak detectable signals), which often requires sophisticated multi-step signal amplification strategies, such as DNA barcodes or enzymatic signal amplification (digital ELISA). Flow cytometry often underestimates EV counts because many small vesicles (<200 nm) could be missed due to their weak light scattering, or a swarm of vesicles could be counted as a single event. More importantly, many of these methods are suboptimal for detecting and quantifying very rare in excessive background EVs and particles. Multiplexed analysis of EVs is especially critical to identify the origin of individual EVs and molecularly profile biomarkers associated with targeted therapies and mutations.

### Idiopathic Pulmonary Fibrosis Markers

Applicant has found that IPF markers MMP-7, CRIP-1, TPPP3, CAMPK1D, GSN, Col1A1, COL3A1, SERPINE1, TREM2, CCL18, SPP-1, α-SMA, and CD206 are highly expressed in epithelial cells, fibroblasts, and macrophages in cases of IPF, such that significant co-localization of IPF marker(s) with cell-specific marker(s) is indicative of detecting IPF in a sample. Therefore, co-localization of MMP-7, CRIP-1, TPPP3, CAMPK1D, GSN, Col1A1, COL3A1, SERPINE1, TREM2, CCL18, SPP-1, α-SMA, and/or CD206 with the cell-specific markers EpCAM, E-cadherin, CD68, CD45, PDGFRα, and/or FAP (e.g., as shown in e.g., Table 1) can be used to diagnose a subject with idiopathic pulmonary fibrosis and/or to determine whether IPF is progressing in a subject.

### Cell-Specific Markers

Extracellular vesicles carry markers which typically indicate the origin of their cell type. For example, EpCAM and E-cadherin can be used to detect EVs originating from epithelial cells. CD68 and/or CD45 can be used to detect EVs originating from macrophages, and PDGFRα, and/or FAP can be used to detect EVs originating from fibroblasts. In addition, there are leukocytes such as T cells (wherein CD2 is a cell-specific marker) and B cells (wherein CD19 is a cell-specific marker). Many EVs secreted into bronchoalveolar lavage fluid, which contains epithelial cells, macrophages, and fibroblasts may contain one or more of these cell-specific markers. Therefore, monitoring EVs that comprise one or more of EpCAM, E-cadherin, CD68, CD45, PDGFRα, and/or FAP, in combination with idiopathic pulmonary fibrosis markers, in a subject can give information regarding a subject's idiopathic pulmonary fibrosis status. Thus herein, we provide methods wherein monitoring specific IPF markers in EVs comprising one or more of EpCAM, E-cadherin, CD68, CD45, PDGFRα, and/or FAP in a subject can be used to diagnose a subject with idiopathic pulmonary fibrosis. See, e.g., Table 1.

### Examples

The invention is further described in the following examples, which do not limit the scope of the invention described in the claims. The methods presented here were used to carry out the examples.

### Methods

### Antibodies

The following antibodies were used in the examples below: anti-EpCAM, CD45 and CD68, MA5-12436, 14-0459-82, and 14-0689-82 (Invitrogen); anti-PDGFRα (3174S), anti-FAP (66562S), and anti-MMP7 (71031S) (Cell Signaling Technology); anti-COL1A1 antibody (NB600-450) (Novus Biologicals); anti-SPP1 (ab8448) (Abcam).

### Cell Lines

Beas-2B, MRC5, and THP-1 cell lines were purchased from ATCC. Beas-2B was grown in BEBM mixed with bronchial epithelial cell growth kit (ATCC). To induce the epithelial mesenchymal transition, the Beas-2B cells were treated with TGF-β 10 ng/ml for 48 h. The MRC5 cell line was cultured in Dulbecco's minimum essential medium (Cyclone) containing 10% fetal bovine serum (FBS) (Gibco, Invitrogen), 100 U/mL penicillin, and 100 μg/mL streptomycin. The MRC5 fibroblast to myofibroblast transition was induced with TGF-β 10 ng/ml for 48 or 72 h. THP-1 cells were grown from Roswell Park Memorial Institute 1640 medium (Gibco, Invitrogen) supplemented with 10% FBS, 100 U/mL penicillin, and 100 μg/mL streptomycin. MO macrophage was induced with phorbol 12-myristate 13-acetate 100 ng/ml for 48 h. To make M2-like macrophages, MO macrophages were treated with IL-4 20 ng/mL and IL-13 20 ng/ml for 48 h. All the cells were grown at 37° C. in 5% CO2 and were free from mycoplasma contamination.

EV Isolation from Conditioned Medium

Once the cell confluence was ~70-80%, the cells were washed with PBS twice and incubated in a medium with 2% exosome-depleted FBS (Thermo Fisher) for 24 h. The conditioned medium was filtered through a 40 μm cell strainer and subsequently through a 0.8 μm PES membrane filter (Thermo Fisher). The medium was concentrated with Centricon Plus-70 Centrifugal Filter (MWCO=10 kDa, Millipore Sigma) at 3,500 g for 30 min at 4° C. Then, the medium was separated by size-exclusion chromatography (SEC), wherein the 4th and 5th fractions were abundant in EV-sized particles. An Amicon Ultra-2 Centrifugal Filter (MWCO=100 kDa, Millipore Sigma) was used to concentrate the EVs from the medium at 3,500 g for 30 min at 4° C. The isolated EVs were aliquoted in tubes and stored in −80° C.

EV Isolation from Bronchoalveolar Lavage Fluid (BALF)

BALF samples (~1 mL) were centrifuged with 300 g for 10 min at 4° C. to get rid of cells or large cell debris followed by 2,000 g for 20 min for removing large EVs. The samples were passed through the SEC, and 4-5th fractions were collected for the further processing. The fractions were centrifuged in the Amicon Ultra-2 Centrifugal Filter at 3,500 g for 30 min at 4° C. The isolated EVs were aliquoted in tubes and stored in −80° C.

### Characterization of EVs

The number of EVs was measured by nanoparticle tracking analysis (Nanosight LM10 microscope, Malvern). The experiment was conducted with a 642 nm laser module at room temperature. Each sample was diluted 500-fold in PBS and manually placed in the chamber. Each experiment was performed for 30 s in quadruplicate. The number of particles per frame was 33.3-87.7, and the frame rate per second was 30.

EVs and EV-liposome fused particles were fixed with 2% paraformaldehyde (PFA, Electron Microscopy Sciences). These were loaded onto formvar carbon-coated EM grids (Ted Pella) and incubated for 20 min. The grids were washed with PBS and transferred to 1% glutaraldehyde, and incubated for 5 min. The grids were then washed with distilled water. Next, the particles were stained with a mixture of 2% uranyl acetate (Electron Microscopy Sciences) and 75 mm oxalic acid (Millipore Sigma) for 1 min. The grids were dried for 5 min at room temperature after the removal of the excessive solution with filter paper. The liposomes were prepared by the same procedure without fixation. Finally, the vesicles were imaged by transmission electron microscopy (Hitachi) at 100 kV.

### Chip Fabrication

Periodic nanowell arrays were patterned (200 nm in diameter and 500 nm in periodicity) using interference lithography (LumArray, Inc., Somerville, MA, USA). Briefly, a 200 nm thick, low-stress silicon nitride layer was deposited by low-pressure chemical vapor deposition (LPCVD) on 4-inch silicon wafers. After anti-reflection coating (ARC) and spin-coating of a negative photoresist, two orthogonal grating images were exposed to the photoresist and made periodic nanowell patterns. Subsequent reactive ion etching with CF4 transferred the hole patterns into the silicon nitride layer. The remaining resists were removed by piranha cleaning. Deposition of 100 nm thick gold with a 5 nm titanium adhesion layer produced periodic gold nanowell arrays. The wafers were then diced into smaller pieces for EV assays.

### SCOPE-IPF Assay

Clean nPLEX chips were functionalized with a mixture of SH-PEG-COOH 1 k (Nanocs) and SH-mPEG 0.35 k at a ratio of 1:3 overnight. EDC (Thermo Scientific) and sulfo-NHS (Thermo Scientific) were used to capture EVs' amine group to the chips. After washing with PBS, EVs were dropped on the chips and incubated for 30 min, followed by fixation and permeabilization. The EVs were blocked with 2% BSA for 30 min and immuno-labeled with primary antibodies for 1 h. The secondary antibody conjugated with AlexaFluor 488, AlexaFluor 555, AlexaFluor 647 or DyLight 755, were incubated with EVs for 30 min. PBS washing was required in every process. The chips were finally mounted on the slide with a mounting solution (ProLong Gold Antifade mounting, Thermo Fisher) and covered with a glass coverslip. The single EV images were taken with an upright fluorescent microscope (Zeiss).

### Image Processing

Images were analyzed using ImageJ (ImageJ2 Fiji, version 2.14.0/1.54h) and custom-built Python (Python 3.9). The background signals were subtracted using a rolling ball algorithm (radium=50) and then the ComDet plugin was used to detect EV location. Based on the EV location, the other fluorescent channels for the markers were measured from 3×3 pixel window. The threshold of each marker was set by fluorescence intensities of the Isotype control antibodies.

### Bead-Based Flow Cytometry

10⁹ to 10¹⁰ EVs were incubated with 0.2 μL aldehyde/sulfate beads (Invitrogen) for 30 min to be attached to the beads. After blocking with 1% BSA in PBS for 2 h and glycine for 30 min, the beads were incubated with primary antibodies diluted in 1% BSA for 1 h, followed by incubation with secondary antibodies conjugated with Alexa Flour 488 for 1 h. The beads were analysis with a CytoFlex flow cytometer (Beckman Coulter) using the 488/8 bandpass filter for FITC. The median fluorescence intensity was analyzed with FlowJo X 10.0. The normalization was done with the isotope control.

### Example 1: High-Resolution nPLEX System Distinguishes Cell-Specific Extracellular Vesicles

Detected particles were plotted in a histogram to compare the intensities and EV counts between nPLEX and glass. nPLEX not only amplifies signals, but it enhances the signal-to-noise (SNR) ratio by decreasing background noise. The end result is that more particles are detected on nPLEX compared to glass: in AF555, AF647, and Cy7 channels, nPLEX showed the higher SNR and more EVs compared to glass.

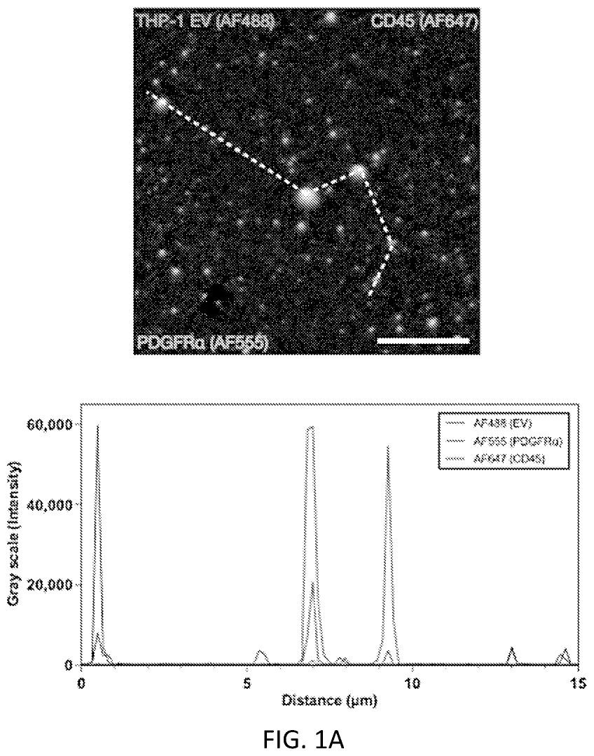

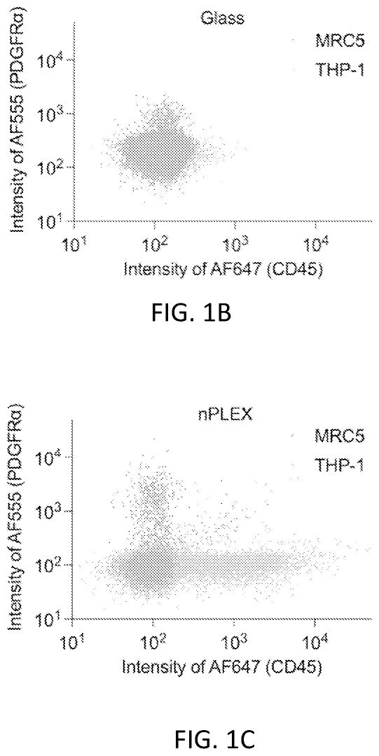

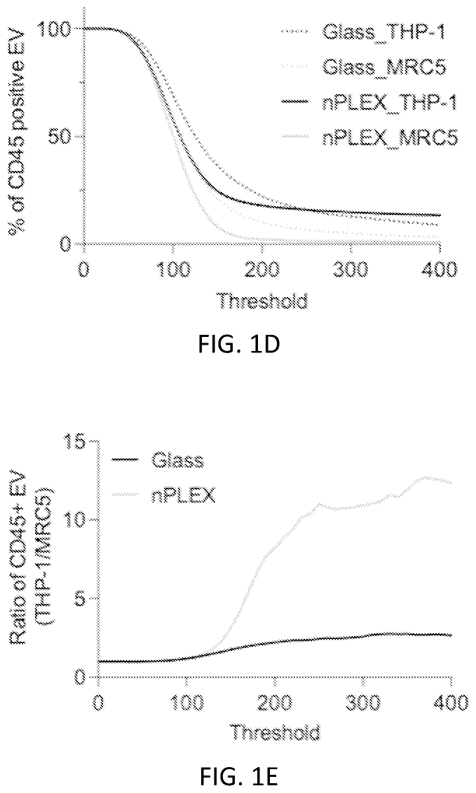

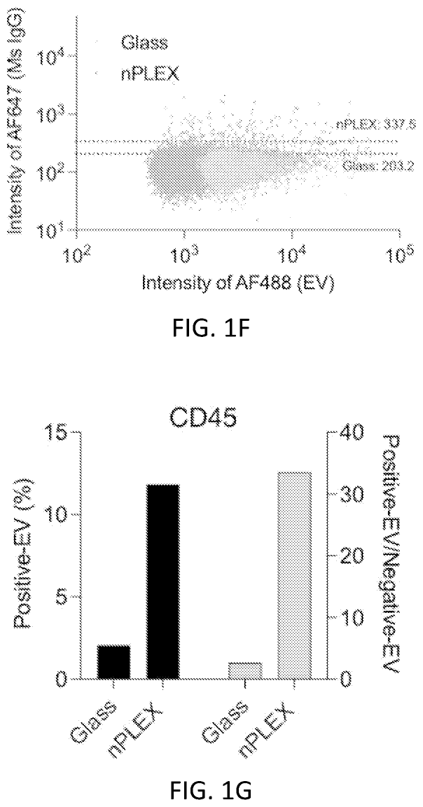

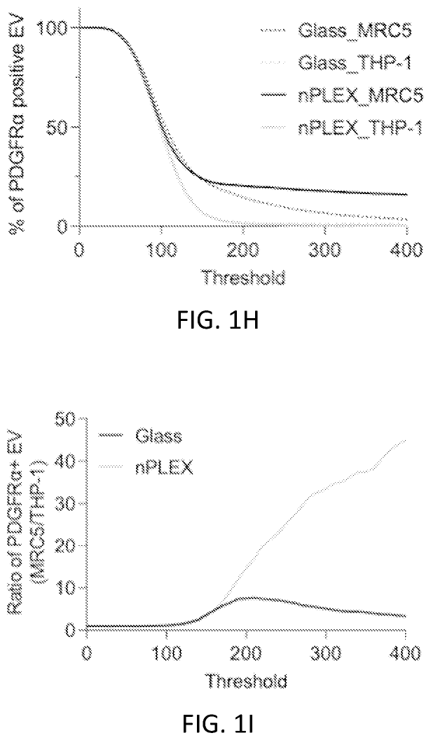

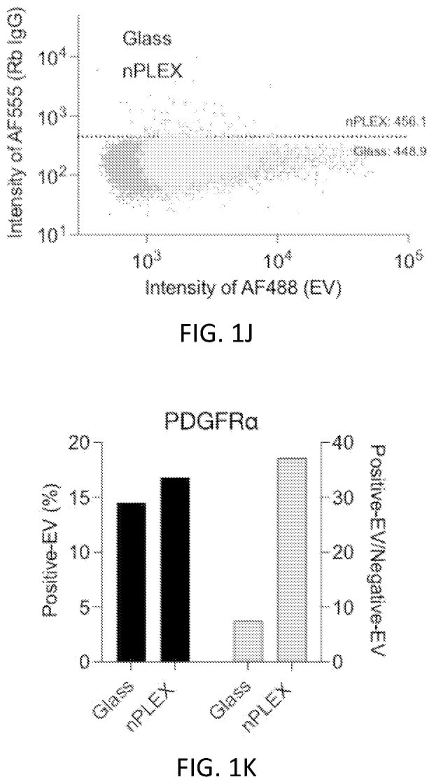

To compare marker intensities between glass and nPLEX, EVs were labeled with AF488 to show the similar number of EVs on glass and nPLEX, and labeled with AF555 and AF647 for PDGFRα and CD45, respectively. After getting the EV location (Jeong et al., 2023), the markers' intensities were measured on that position (FIG. 1A). The quantified two values on each spot including AF555 and AF647 were plotted (FIGS. 1B and 1C). The EVs derived from MRC5 which is a lung fibroblast, PDGFRα-positive and CD45-negative cell, expressed higher value in PDGFRα compared to the EVs from THP-1 which is a macrophage, CD45-positive and PDGFRα-negative cell. On the nPLEX, the different expressions could be amplified 10 times more than glass (FIGS. 1B and 1C), which resulted in greater distinction between two types of EVs. To demonstrate that the distinguishability is enhanced in nPLEX, the threshold was changed and followed the expression of each marker in each type of EVs (FIGS. 1D and 1H). Moreover, the marker signals from marker-negative cells' EVs were considered noise and divided the marker-positive EVs percentage with the negative ones. The ratio increased consistently in nPLEX because nPLEX had lower signal in marker-negative EVs and higher signal in marker-positive EVs (FIGS. 1E and 1I). Considering the IgG isotope value, we could have the baseline graph (FIGS. 1F and 1J). The maximum marker expression values were shown, which was set by the signal of IgG and marker-negative EVs (FIGS. 1G and 1K). Thus, in the terms of EVs and markers, the background signals were lower and the actual signals were higher on the nPLEX gold chips, which generated advanced ability to distinguish different types of EVs.

### Example 2: Specificity of Cell-Specific Markers in EVs

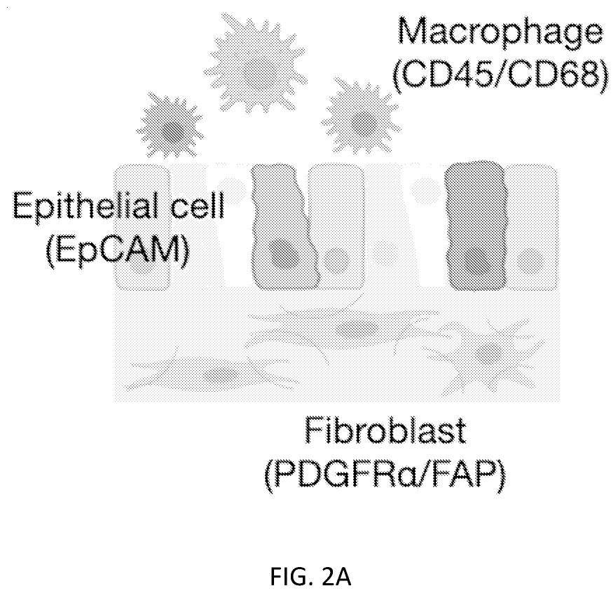

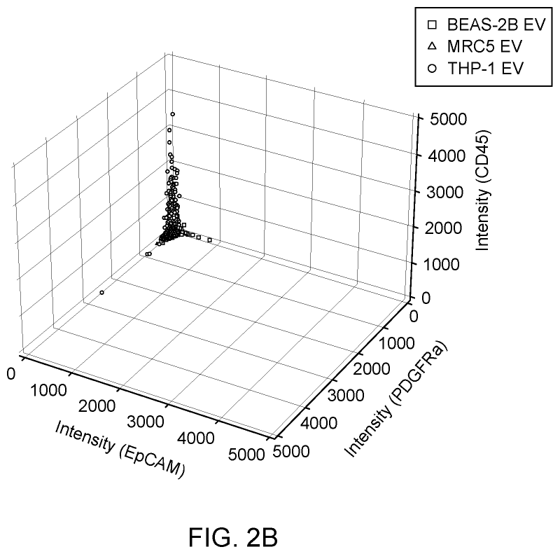

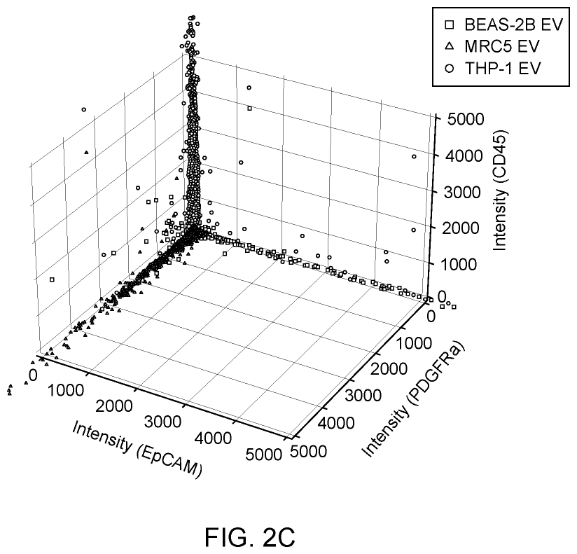

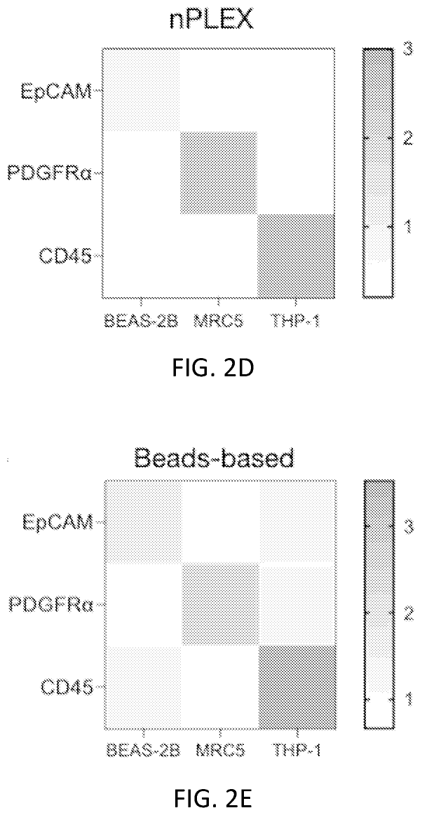

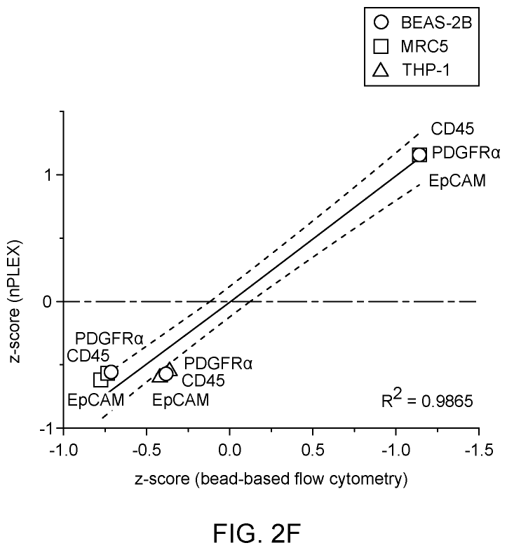

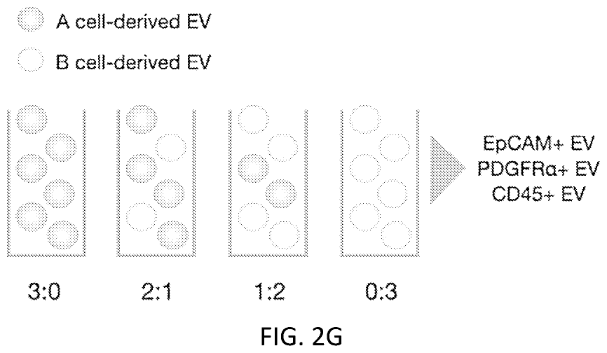

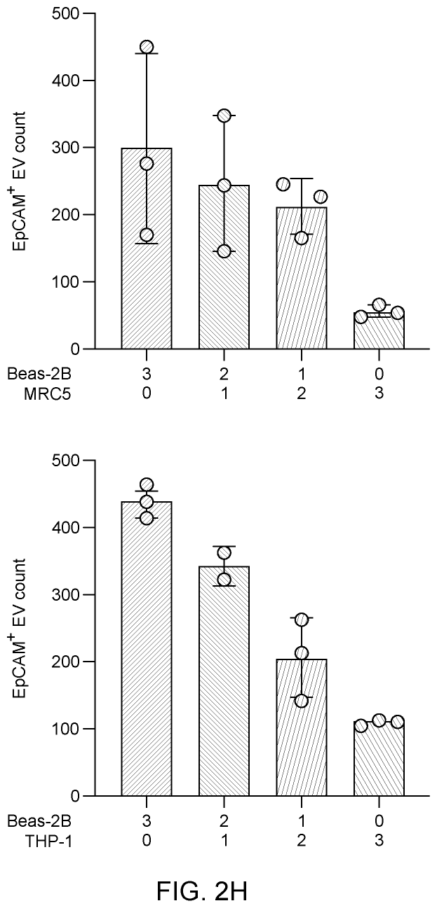

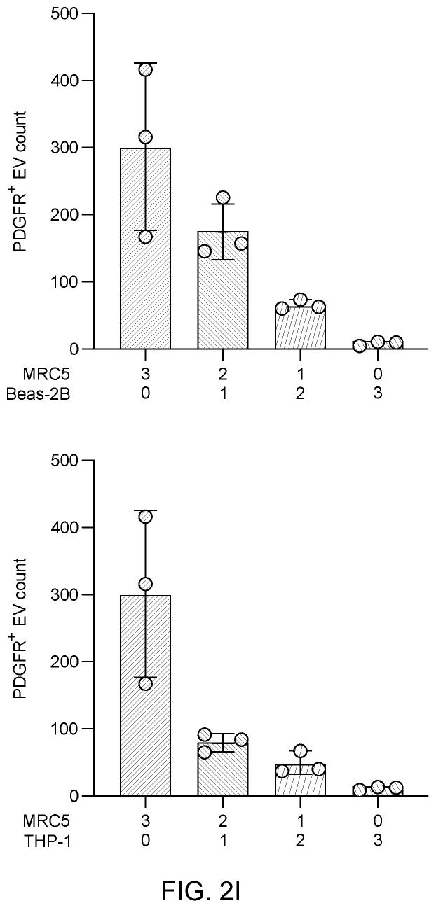

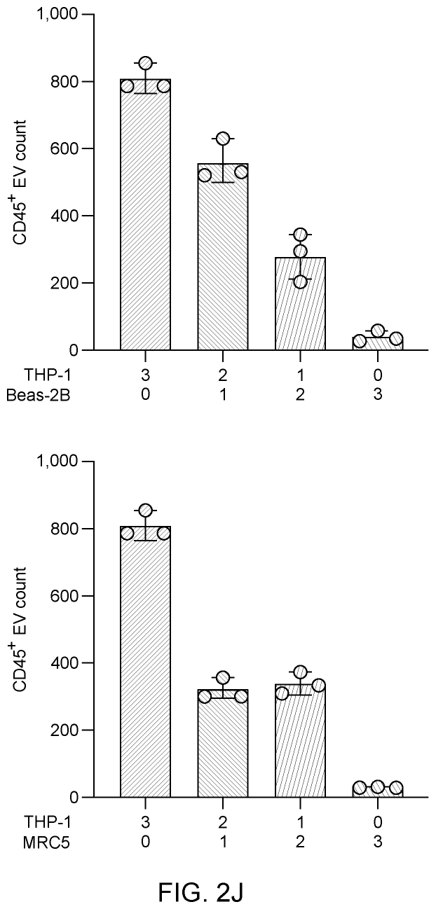

The nPLEX system was used to detect cell-specific markers from epithelial cells, fibroblasts, and macrophages, each of which are implicated in IPF. BEAS-2B (a bronchial epithelial cell line), MRC5 (a lung fibroblast cell line), and THP-1 (a macrophage cell line) were used to elucidate cell-specific EV biomarkers that could be used to determine IPF. After isolating EVs from the conditioned medium of each of these cell lines, the EVs were visualized with Transmission Electron Microscopy (TEM) after negative staining (FIG. 2A). For characterization, the sizes and numbers of EVs were measured with dynamic light scattering (DLS) and nanoparticle tracking analysis (NTA). The average sizes of EVs were ~200 nm. Each cell lines exhibits specific markers: BEAS-2B EVs express EpCAM and E-cadherin, MRC5 EVs express PDGFRα and FAP, and THP-1 EVs express CD45 and CD68. The specific markers of the EVs were evaluated with bead-based flow cytometry and western blot which were the bulk EV analysis. To verify the specificity of the antibodies for the specific markers, each type of EVs were incubated with the antibody cocktails including anti-EpCAM (Cy7), anti-PDGFRα (AF555), and anti-CD45 (AF647) on glass (FIG. 2B) and nPLEX (FIG. 2C). The antibodies labeled the EVs having their specific markers. The markers' intensities in all channels were amplified on nPLEX on the 3D graph. Each marker was distinctly expressed in its specific cells' EVs in nPLEX (FIG. 2D), and bead-based flow cytometry (FIG. 2E). Between nPLEX and bead-based flow cytometry, the correlation value (R²=0.9865) demonstrated that nPLEX could measure the marker expression with the similar values of conventional method (FIG. 2F).

As a clinical sample, bronchoalveolar lavage fluid (BALF), contains EVs derived from many sources. Specific mixtures of EVs from different parental cells were prepared and evaluated for cell-specific markers. Fewer EVs of each specific-cell type (FIG. 2G), corresponded with a proportional decrease in the markers detected (FIGS. 2H, 2I, and 2J, for EpCAM, PDGFRα, and CD45, respectively), which meant that the antibodies could selectively capture the markers.

### Example 3: Analysis of Double-Positive EVs

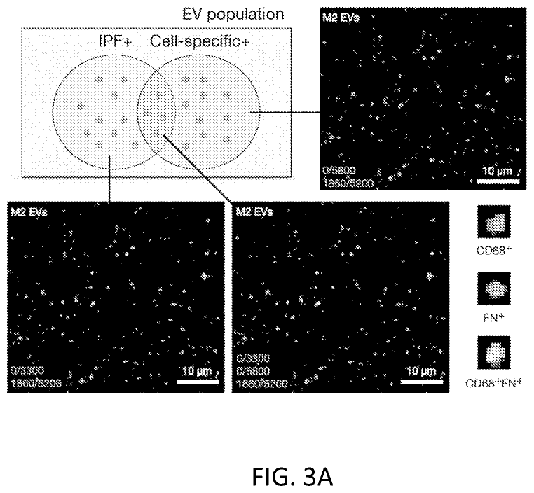

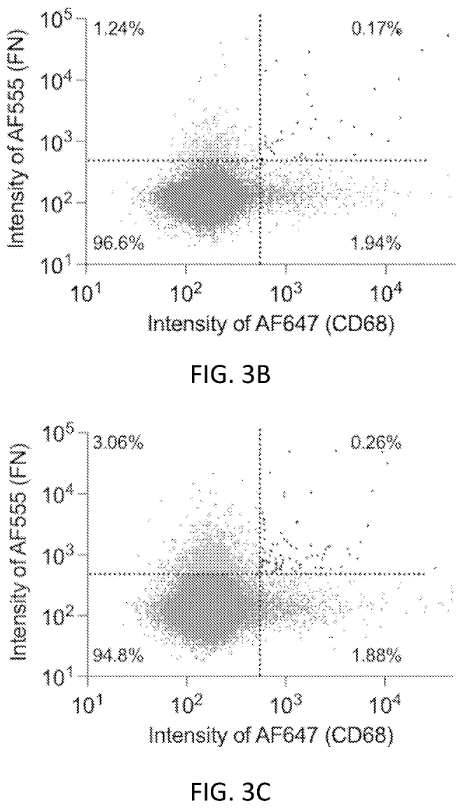

Next, double labeling of IPF markers and cell-specific markers on single EV was performed. The antibody double-labeled EVs were evaluated by marker intensities. First, the location information from EV channel (Cy7) was collected, then the intensities of cell-specific marker (AF555) and IPF marker (AF647) were measured (FIG. 3A). The measured values of markers were plotted; first quadrant was for double-positive EVs, second quadrant meant for IPF marker-positive EVs, and fourth quadrant was for cell-specific marker-positive EVs (FIGS. 3B and 3C). The threshold was set with EVs labeled with the IgG isotype antibodies. As more than one antibody was used, the steric hindrance was checked by comparing the percentage of positive EVs when single-labeling and double-labeling. The antibody combination did not show the steric hindrance even when labeling with two antibodies. The fibronectin and CD68 on EVs from MO (MO EVs) and M2-like macrophages (M2 EVs) were compared. The fibronectin-positive EVs increased in M2 EVs. To confirm the data, the single EV flow cytometry was repeated and double-positive EVs in M2 had higher percentages than MO in flow cytometry as well. This shows the double-labeling system is comparable with the conventional EV labeling method.

### Example 4: Examination of Double-Positive EVs for Detection of IPF

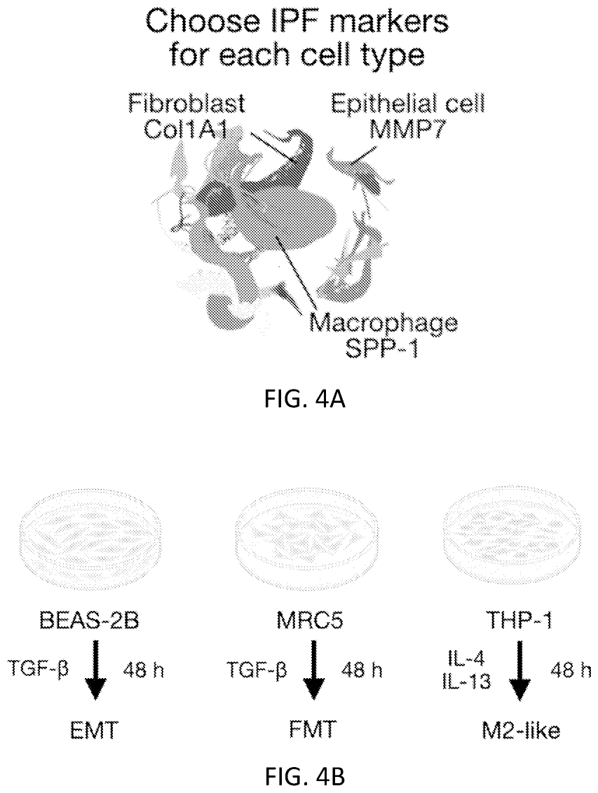

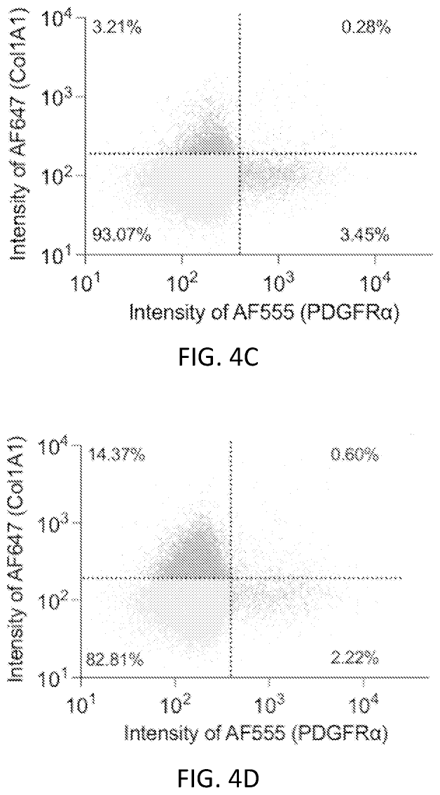

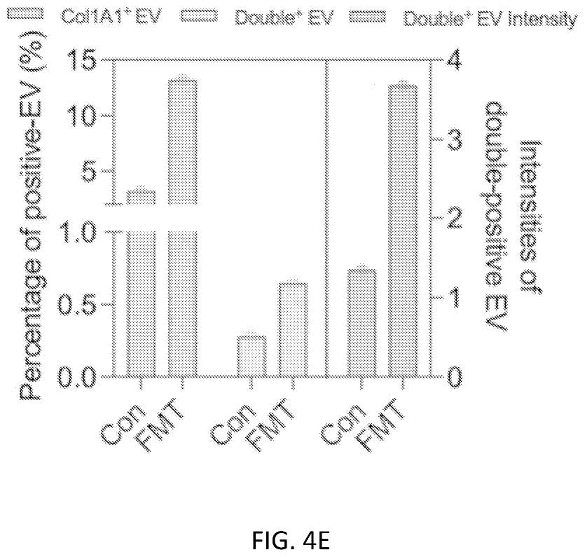

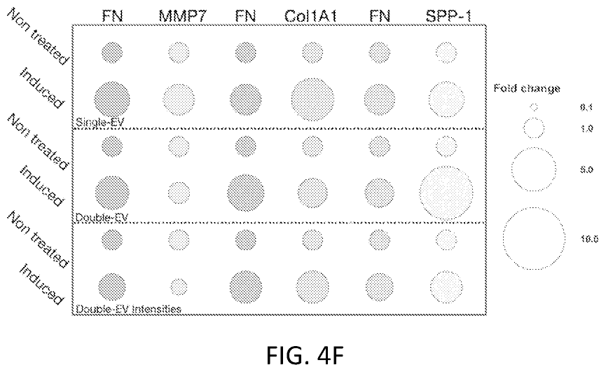

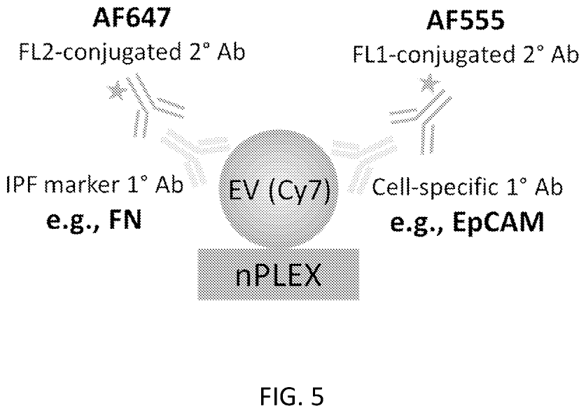

As mentioned above, three key cell types are implicated in IPF including epithelial cells, fibroblasts, and macrophages. To select the IPF markers for each type of cells, we reanalyzed the single cell-sequencing data from IPF Cell Atlas (ipfcellatlas.com) (FIG. 4A). We used five published data and chose 30 top genes from the data. MMP-7, Col1A1, and SPP-1 were the most intersected markers in five studies for epithelial cells, fibroblasts, and macrophages, respectively (FIG. 5A). To validate the antibodies for our system, we made marker-overexpressing in vitro models including epithelial-mesenchymal transition, fibroblast-myofibroblast transition, and M2-like macrophage to isolate marker-positive EVs (FIG. 4B). After isolating the EVs, we confirmed the increased level of each marker using western blot. Not only selected IPF markers, fibrosis-associated marker, fibronectin was also increased. We evaluated the single- and double-positive EVs from these EVs (FIGS. 4C and 4D). Col1A1-overexpressing EVs showed higher percentages in the first and second quadrant which indicates double- and single-positive EVs. From the intensities of double-positive EVs, we could get the differential expression between control and marker-overexpressing EVs (FIG. 4E). With the same methods, we could evaluate the single- and double-positive EVs derived from three in vitro models (FIG. 4F).

### Example 5: Data of BALF Samples

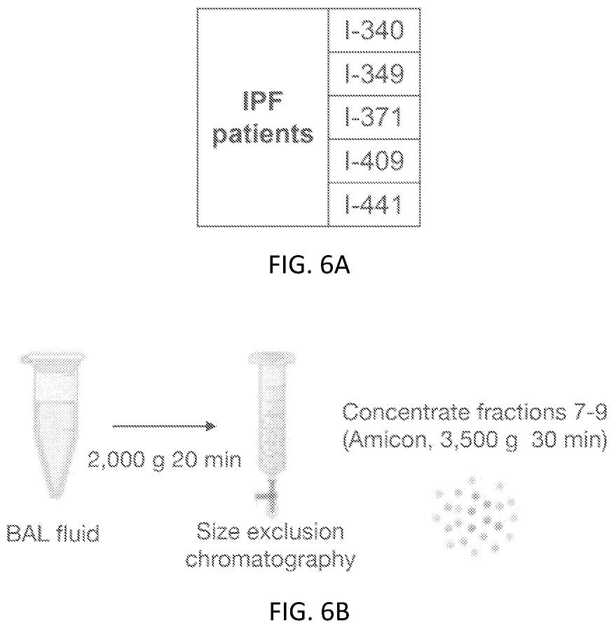

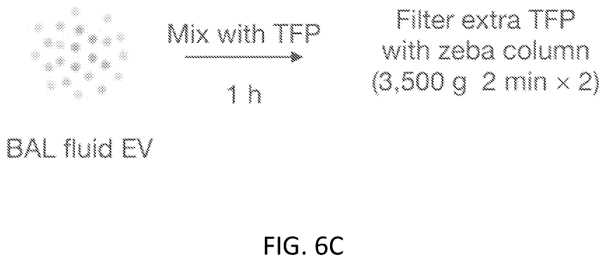

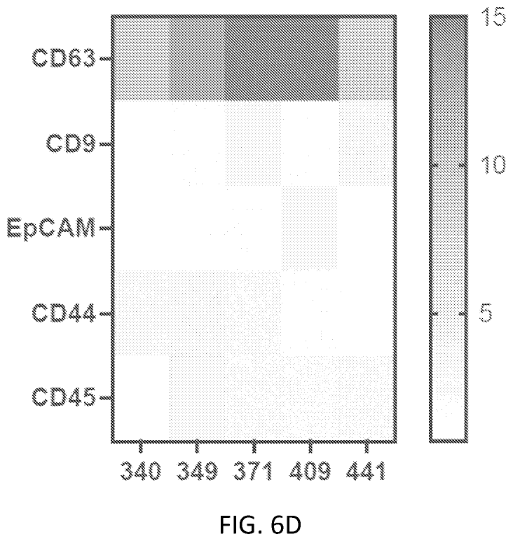

Bronchoalveolar lavage fluid (BALF) was collected from five IPF patients (FIG. 6A). EVs were isolated from the BALF samples and were fluorescently labeled for EV quantification (FIG. 6B and FIG. 6C). Specifically, the BALF samples were centrifuged at 2,000 g for 20 min to remove large particles from BALF. Then, size exclusion chromatography was applied to isolate the EVs based on EVs' unique sizes. The fractions containing EVs were condensed with Amicon Filter with 3,500 g for 30 min. The isolated EVs were then captured on a nanoplasmonic chip and immunolabeled for cell-specific markers. Cell-specific EV markers (e.g., EpCAM, PDGFRα, and CD45) were measured and represented as a heatmap after quantification (FIG. 6D).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A non-invasive method of detecting idiopathic pulmonary fibrosis in a subject, the method comprising:
(a) isolating, detecting, or enriching extracellular vesicles (EVs) from a sample obtained from a subject;
(b) labeling the EVs with one or more antibodies or antigen binding portions thereof that bind to one or more idiopathic pulmonary fibrosis marker(s) and one or more antibodies or antigen binding portions thereof that bind to one or more cell-specific marker(s), preferably wherein the markers are as shown in Table 1;
(c) determining the counts of EVs positive for colocalized idiopathic pulmonary fibrosis marker(s) and cell-specific marker(s) or determining the intensity levels of idiopathic pulmonary fibrosis marker(s) and cell-specific marker(s) expressed in EVs.

2. The method of claim 1, further comprising using a plasmon-enhanced EV detection method in step (b).

3. The method of claim 1, wherein the antibodies or antigen binding portions thereof that bind to the EV idiopathic pulmonary fibrosis marker(s) comprise fluorescent dyes.

4. The method of claim 1, wherein the one or more antibodies or antigen binding portions thereof that bind the one or more idiopathic pulmonary fibrosis marker(s) comprise one or more antibodies or antigen binding portions thereof that bind to matrix metallopeptidase 7 (MMP-7), cysteine-rich intestinal protein (CRIP-1), tubulin polymerization promoting protein family member 3 (TPPP3), calcium/calmodulin-dependent protein kinase 1 (CAMPK1D), gelsolin (GSN), collagen, type I, alpha 1 (Col1A1), pro-alpha1 chains of type III collagen (COL3A1), plasminogen activator inhibitor 1 (SERPINE1), chemokine ligand 18 (CCL18), triggering receptor expressed on myeloid cells 2 (TREM2), secreted phosphoprotein 1 (SPP-1), alpha-smooth muscle actin (α-SMA), and/or CD206.

5. The method of claim 1, wherein the one or more antibodies or antigen binding portions thereof that bind the one or more cell-specific marker(s) comprise one or more antibodies or antigen binding portions thereof that bind to epithelial cellular adhesion molecule (EpCAM), E-cadherin, platelet-derived growth factor receptor alpha (PDGFRα), fibroblast activation protein (FAP), CD45, and/or CD68.

6. The method of claim 1, wherein the determining the counts of EVs positive for of idiopathic pulmonary fibrosis marker(s) and cell-specific marker(s) comprises quantification of expression, concentration, intensity, or colocalization.

7. The method of claim 1, wherein the quantification of expression, concentration, intensity, or colocalization are analyzed using multichannel fluorescence imaging in a single EV.

8. The method of claim 1, wherein the sample comprises bronchoalveolar lavage fluid.

9. A non-invasive method of diagnosing idiopathic pulmonary fibrosis in a subject, the method comprising:
(a) isolating, detecting, or enriching extracellular vesicles (EVs) from a sample obtained from a subject, preferably wherein the EVs are labeled with one or more antibodies or antigen binding portions thereof that bind to one or more idiopathic pulmonary fibrosis marker(s) and one or more antibodies or antigen binding portions thereof that bind to one or more cell-specific marker(s), preferably wherein the markers are as shown in Table 1;
(b) determining the counts of EVs positive for colocalized idiopathic pulmonary fibrosis marker(s) and cell-specific marker(s) or determining the intensity levels of idiopathic pulmonary fibrosis marker(s) and cell-specific marker(s) expressed in EVs; and
(c) comparing the counts of EVs or the marker intensity levels determined in step (b) to a reference level, wherein the counts of EVs or the marker intensity levels determined in step (b) greater than the reference level indicate whether the subject has idiopathic pulmonary fibrosis.

10. The method of claim 9, further comprising using a plasmon-enhanced EV detection method in step (b).

11. The method of claim 9, wherein the antibodies or antigen binding portions thereof that bind to the EV idiopathic pulmonary fibrosis marker(s) comprise fluorescent dyes.

12. The method of claim 9, wherein the one or more antibodies or antigen binding portions thereof that bind the one or more idiopathic pulmonary fibrosis marker(s) comprise one or more antibodies or antigen binding portions thereof that bind to matrix metallopeptidase 7 (MMP-7), cysteine-rich intestinal protein (CRIP-1), tubulin polymerization promoting protein family member 3 (TPPP3), calcium/calmodulin-dependent protein kinase 1 (CAMPK1D), gelsolin (GSN), collagen, type I, alpha 1 (Col1A1), pro-alpha1 chains of type III collagen (COL3A1), plasminogen activator inhibitor 1 (SERPINE1), chemokine ligand 18 (CCL18), triggering receptor expressed on myeloid cells 2 (TREM2), secreted phosphoprotein 1 (SPP-1), alpha-smooth muscle actin (α-SMA), and/or CD206.

13. The method of claim 9, wherein the one or more antibodies or antigen binding portions thereof that bind the one or more cell-specific marker(s) comprise one or more antibodies or antigen binding portions thereof that bind to EpCAM, E-cadherin, PDGFRα, FAP, CD45, and/or CD68.

14. The method of claim 9, wherein the determining the counts of EVs positive for idiopathic pulmonary fibrosis marker(s) and cell-specific marker(s) comprises quantification of expression, concentration, intensity, or colocalization.

15. The method of claim 9, wherein the quantification of expression, concentration, intensity, or colocalization are analyzed using multichannel fluorescence imaging in a single EV.

16. The method of claim 9, wherein the sample comprises bronchoalveolar lavage fluid.

17. A method for monitoring idiopathic pulmonary fibrosis in a subject over time, the method comprising:
(a) isolating extracellular vesicles (EVs) in a first sample obtained from a subject at a first time point, wherein isolating EVs comprises:
(i) applying the first sample to a functionalized substrate to capture extracellular vesicles (EVs);
(ii) labeling the EVs with an antibody or antigen binding portions thereof that bind to a previously selected idiopathic pulmonary fibrosis marker(s); and
(iii) labeling the EVs with an antibody or antigen binding portions thereof that bind to previously a selected cell-specific marker(s); preferably wherein the idiopathic pulmonary fibrosis markers and cell-specific markers are as shown in Table 1,
(b) determining a count of EVs positive for the co-localization of cell-specific marker(s) and idiopathic pulmonary fibrosis marker(s) at a first time point;
(c) isolating EVs in a second sample from the subject obtained at a second time point,
wherein the isolating EVs comprises:
(i) applying the second sample to a functionalized substrate to capture EVs;
(ii) labeling the EVs with an antibody or antigen binding portions thereof that bind to the previously selected idiopathic pulmonary fibrosis marker(s) of step (a); and
(iii) labeling the EVs with an antibody or antigen binding portions thereof that bind to previously a selected cell-specific marker(s) of step (a);
(d) determining a count of EVs positive for the co-localization cell-specific marker(s) and idiopathic pulmonary fibrosis marker(s) at a second time point; thereby monitoring idiopathic pulmonary fibrosis in a subject over time.

18. The method of claim 17, further comprising using a plasmon-enhanced EV detection method in step (b) and/or step (d).

19. The method of claim 17, wherein the antibodies or antigen binding portions thereof that bind to the EV idiopathic pulmonary fibrosis marker(s) comprise fluorescent dyes.

20. The method of claim 17, wherein the one or more antibodies or antigen binding portions thereof that bind the one or more idiopathic pulmonary fibrosis marker(s) comprise one or more antibodies or antigen binding portions thereof that bind to matrix metallopeptidase 7 (MMP-7), cysteine-rich intestinal protein (CRIP-1), tubulin polymerization promoting protein family member 3 (TPPP3), calcium/calmodulin-dependent protein kinase 1 (CAMPK1D), gelsolin (GSN), collagen, type I, alpha 1 (Col1A1), pro-alpha1 chains of type III collagen (COL3A1), plasminogen activator inhibitor 1 (SERPINE1), chemokine ligand 18 (CCL18), triggering receptor expressed on myeloid cells 2 (TREM2), secreted phosphoprotein 1 (SPP-1), alpha-smooth muscle actin (α-SMA), and/or CD206.

21. The method of claim 17, wherein the one or more antibodies or antigen binding portions thereof that bind the one or more cell-specific marker(s) comprise one or more antibodies or antigen binding portions thereof that bind to EpCAM, E-cadherin, PDGFRα, FAP, CD45, and/or CD68.

22. The method of claim 17, wherein the determining the counts of EVs positive for idiopathic pulmonary fibrosis marker(s) and cell-specific marker(s) comprises quantification of expression, concentration, intensity, or colocalization.

23. The method of claim 17, wherein the quantification of expression, concentration, intensity, or colocalization are analyzed using multichannel fluorescence imaging in a single EV.

24. The method of claim 17, wherein the sample comprises bronchoalveolar lavage fluid.

25. The method of claim 9, wherein the presence of a level of EVs with co-localization of idiopathic pulmonary fibrosis marker(s) and cell-specific marker(s) above a reference level indicates that the subject has idiopathic pulmonary fibrosis.

26. The method of claim 25, further comprising identifying a subject who has a level of EVs with co-localization of idiopathic pulmonary fibrosis marker(s) and cell-specific marker(s) above a reference level, and administering a treatment for idiopathic pulmonary fibrosis to the subject.

27. The method of claim 26, wherein the treatment comprises administration of supplemental oxygen therapy, an antifibrotic therapy, and/or a tyrosine kinase inhibitor.

28. The method of claim 17, wherein the method further comprises administering at least one dose of a treatment or candidate treatment for idiopathic pulmonary fibroses to the subject between steps (b) and (c).

29. The method of claim 28, wherein the treatment comprising administration of supplemental oxygen therapy, an antifibrotic therapy, and/or a tyrosine kinase inhibitor.
